(19) United States
(12) Patent Application Publication
(10) Pub. No.: US 2026/0193596 A1
(43) Pub. Date: Jul. 9, 2026

(54) ODOR-PRODUCING ACTINOMYCETE AND APPLICATION THEREOF

(71) Applicant: Xi’an University of Architecture and Technology, Xi'an (CN)

(72) Inventors: Haihan Zhang, Xi'an (CN); Xiang Liu, Xi'an (CN); Ben Ma, Xi'an (CN); Tongchao Ni, Xi'an (CN)

(21) Appl. No.: 19/330,935

(22) Filed: Sep. 17, 2025

(30) Foreign Application Priority Data
Jan. 6, 2025 (CN) 2025100120463

(51) Int. Cl. C12N 1/20; C12R 1/04

(52) U.S. Cl. CPC C12N 1/205; C12R 2001/04

(57) ABSTRACT

An odor-producing actinomycete and an application thereof are provided, relating to the technical field of microorganisms. Strains of the odor-producing actinomycete have strong odor-producing capacity. During a germination stage of the strains, FSC increases from 2594 at 0 h to 3644 at 5 h, and a concentration of 2-MIB increases from 6.3144 ng/L at 0 h to 30.4838 ng/L at 5 h; therefore, the strains of the odor-producing actinomycete can be used to determine sources of odor issues of water sources.

### CROSS-REFERENCE TO RELATED APPLICATION

This application claims priority to Chinese Patent Application No. 202510012046.3, filed on Jan. 6, 2025, which is herein incorporated by reference in its entirety.

### TECHNICAL FIELD

The disclosure relates to the technical field of microorganisms, and more particularly to an odor-producing actinomycete and an application thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 25031MYZ-USP1-SL. The XML file is 5113 bytes; is created on Aug. 25, 2025; and is being submitted electronically via patent center.

### BACKGROUND

Odor issues, especially earthy and moldy smells occurring in source water reservoirs, have become a serious challenge to drinking water safety. Geosmin and 2-methylisoborneol (2-MIB) are primary odor-causing compounds, featured by their extremely low human odor detection thresholds. According to a Chinese national standard “Hygienic Standard for Drinking Water” GB 5749-2022, limit values of these two odor-causing compounds in drinking water are only 10 nanograms per liter (ng/L). Furthermore, the standard specifies that the drinking water should have good sensory properties without odors and unusual tastes. Aquatic microorganisms such as cyanobacteria, actinomycetes, myxobacteria, and fungi in natural water will release a series of secondary metabolites during seasonal reproduction, including these two odor-causing compounds. The cyanobacteria are considered main source of these two odor-causing compounds, which has attracted attention of scholars. However, in a non-cyanobacterial outbreak stage, researches on odor-causing compounds mainly secreted by the actinomycetes are often ignored. Identifying sources of the odor issues is of great significance for target prevention and control of the odor issues. Compared with algae, odor production of the actinomycetes reduces light demand, and the actinomycetes have a wide spatial distribution and are not limited to shallow layers of reservoirs. The actinomycetes in natural water can be divided into two states: active hyphae and resting spores. Actinomycete spores have strong adaptability and tolerance to extreme environmental conditions and need to germinate and grow into hyphae under suitable conditions to produce the odor-causing compounds. This process is usually random and regulated by various objective factors such as temperature, nutrients, and potential of hydrogen (pH). Due to this unpredictability, opportunities to early intervene in odor events caused by germination of the actinomycete spores are often lost. Therefore, it is essential to provide an actinomycete with strong odor-producing capacity as a detection target to determine the sources of the odor issues in water sources.

### SUMMARY

Based on the above technical problems, the disclosure provides an odor-producing actinomycete and an application thereof.

The odor-producing actinomycete is Streptomyces antibioticus LJH21, deposit in China Center for Type Culture Collection (CCTCC) with a deposit number of CCTCC No. M 20241163, on Jun. 6, 2024, at Wuhan University, Wuhan, Hubei Province, China.

The disclosure further provides the application of the odor-producing actinomycete in identifying sources of odor issues in water sources.

In an embodiment, water to be tested is cultured in a solid microbial medium containing potassium dichromate at a constant temperature of 30 degrees Celsius (° C.) to obtain a pre-tested substance. The pre-tested substance is cultured in a spore-producing medium at the constant temperature of 30° C. to obtain a pre-germinated substance, and the pre-germinated substance is germinated and cultured in a liquid microbial medium at a temperature of 30° C. During germinating and culturing the pre-germinated substance, germination determination is performed to determine germination of the odor-producing actinomycete. At the same time, during germination of the pre-germinated substance, a content of 2-MIB is detected. When the content of the 2-MIB increases, the odor-producing actinomycete is one of the sources of the odor issues in the water sources.

A formula of the solid microbial medium includes 20 grams (g) of soluble starch, 1 g of potassium nitrate, 0.5 g of dipotassium hydrogen phosphate, 0.5 g of magnesium sulfate, 0.5 g of sodium chloride, 0.01 g of ferrous sulfate, 15 g of agar, and water to make up to a constant volume of 1 liter (L).

Each liter of the solid microbial culture medium is added with 80 milligrams (mg) of the potassium dichromate.

A formula of the liquid microbial medium includes 20 g of the soluble starch, 1 g of the potassium nitrate, 0.5 g of the dipotassium hydrogen phosphate, 0.5 g of the magnesium sulfate, 0.5 g of the sodium chloride, 0.01 g of the ferrous sulfate, and the water to make up to a constant volume of 1 L.

A formula of the spore-producing medium includes 20 g of oat flour, 0.001 g of the ferrous sulfate, 0.001 g of manganese chloride, 0.001 g to 0.0012 g of zinc sulfate, 1.29 g of calcium chloride, 0.95 g of magnesium chloride, and the water to make up to a constant volume of 1 L.

In an embodiment, a method for the germination determination includes the following steps: germinating and culturing the pre-germinated substance in the liquid microbial medium at 30° C. to obtain a mixed actinomycete bacterial liquid, mixing the mixed actinomycete bacterial liquid with a stain of a flow cytometry, followed by heating in dark to obtain a stained bacterial liquid, and analyzing the stained bacterial liquid to obtain a germination speed of actinomycete spores. Methods for the germination determination of spores in related art mostly depend on conventional optical microscope observation, which has low efficiency and strong subjectivity, significantly prolonging detection times, thereby causing loss of an opportunity to early intervene in odor events caused by germination of the actinomycete spores. By using the odor-producing actinomycete and the application thereof provided by the disclosure, germination state of the actinomycete spores can be simply and efficiently identified.

In an embodiment, a volume ratio of the mixed actinomycete bacterial liquid to the stain is 195:5.

In an embodiment, the stain is a mixed solution of SYBR® Green I and dimethyl sulfoxide, and a volume ratio of the SYBR® Green I to the dimethyl sulfoxide is 5:495.

In an embodiment, parameters for the heating in dark include a heating temperature of 35° C. and a heating duration of 15 minutes (min).

In an embodiment, the content of 2-MIB is detected through a gas chromatography-mass spectrometry (GC-MS).

In an embodiment, detecting parameters for the gas chromatography (GC) include a chromatographic column of DB-WAX, a length of the chromatographic column of 30 meters (m), an inner diameter of the chromatographic column of 250 micrometers (μm), a film thickness of 0.25 μm, a starting temperature of 40° C. maintained for 2 min and then raised at a rate of 10 degrees Celsius per minute (° C./min) to 240° C. maintained for 3 min, a gasification chamber temperature of 250° C., a transmission line temperature of 240° C., a carrier gas of helium (He), a velocity of the carrier gas of 1.0 milliliter per minute (mL/min), and an injection mode of splitless injection. Parameters for the mass spectrometry (MS) include an ion source of electron impact (EI), an electron energy of 70 electron volts (eV), an ion source temperature of 230° C., a quadrupole temperature of 150° C., a scanning mode of Scan, and a scanning mass range of 35 atomic mass units (u) to 550 u.

Compared with the related art, the disclosure has the following beneficial effects.

The odor-producing actinomycete provided by the disclosure is the Streptomyces antibioticus LJH21 deposit in the China Center for Type Culture Collection with a deposit number of CCTCC No. M 20241163. Metabolites of the odor-producing actinomycete include geosmin and the 2-MIB. During a germination stage of actinomycete strains, a cell particle size of the odor-producing actinomycete increases from 2594 at 0 hours (h) to 3644 at 5 h, with a relative growth rate of 40.5%. The concentration of the 2-MIB increases from 6.3144 ng/L at 0 h to 30.4838 ng/L at 5 h; therefore, the odor-producing actinomycete has excellent capacity to produce the 2-MIB.

By using the odor-producing actinomycete capable of producing the 2-MIB as a detection target, the sources of the odor issues in the water sources can be identified, which is conducive to seizing the opportunity to early intervene in the odor events, thereby effectively addressing the odor issues in the water sources as soon as possible and ensuring water source safety.

### BRIEF DESCRIPTION OF DRAWINGS

FIG. 1 illustrates a phylogenetic tree of actinomycete strains according to an embodiment of the disclosure.

FIGS. 2A-2C illustrate a germination determination diagram of actinomycete spores. Specifically, FIG. 2A illustrates a cell granularity range diagram of the actinomycete spores analyzed by a flow cytometry, FIG. 2B illustrates a comparison diagram of cell particle sizes of the actinomycete spores germinated for 0 h and 5 h, and FIG. 2C illustrates a change trend of the cell particle sizes and a relative growth rate diagram of the actinomycete spores germinated for 0 h to 5 h, where SSC represents a cell granularity, FSC represents a cell particle size, and an “A” following the SSC and the FSC is an abbreviation of “area”, representing a way to read data.

FIGS. 3A-3B illustrate a comparison diagram of the cell granularity range of the actinomycete spores germinated for 0 h and 5 h. Specifically, FIG. 3A illustrates a cell granularity range diagram of the actinomycete spores germinated for 0 h, and FIG. 3B illustrates a cell granularity range diagram of the actinomycete spores germinated for 5 h, where SSC represents the cell granularity, and an “A” following the SSC is the abbreviation of “area”, representing a way to read data.

FIG. 4 illustrates a line chart of a concentration of 2-MIB produced by an actinomycete during spore germination.

FIGS. 5A-5B illustrate a comparison of cell morphology of the actinomycete spores germinated for 0 h and 5 h based on a scanning electron microscope. Specifically, FIG. 5A illustrates a cell morphology diagram of the actinomycete spores germinated for 0 h, and FIG. 5B illustrates a cell morphology diagram of the actinomycete spores germinated for 5 h.

### DETAILED DESCRIPTION OF EMBODIMENTS

Embodiments of the disclosure will be described in detail as follows, but it should be understood that, a scope of protection of the disclosure is not limited by the embodiments of the disclosure. Based on the embodiments of the disclosure, all other embodiments obtained by those skilled in the art without creative labor shall fall within the scope of protection of the disclosure. Experiment methods in the disclosure, unless otherwise specified, are conventional methods.

### Embodiment 1

A method for screening an odor-producing actinomycete includes the following steps (1) through (6).

Step (1), a solid medium for enrichment and cultivation of actinomycetes is prepared. Each liter of the solid medium includes the following substances: 20 g of soluble starch, 1 g of potassium nitrate, 0.5 g of dipotassium hydrogen phosphate, 0.5 g of magnesium sulfate, 0.5 g of sodium chloride, 0.01 g of ferrous sulfate, and 15 g of agar. These substances are added into a 1000 milliliters (mL) volumetric flask, and ultrapure water is added to the 1000 mL volumetric flask to make up to a constant volume to thereby obtain a first mixed solution. The first mixed solution is poured into a 1 L sterile conical flask, and then a pH of the first mixed solution is adjusted to 7.4 to obtain a medium. The medium is sterilized at 121° C. for 30 min in an autoclave to obtain a sterilized medium. 80 mg of potassium dichromate is added to the sterilized medium to avoid growth of bacteria and fungi to thereby obtain the solid medium for the enrichment and cultivation of the actinomycetes. The solid medium is cooled to 55° C. and then prepared into a solid medium plate.

A liquid medium for the enrichment and cultivation of the actinomycetes is prepared. Difference between the liquid medium and the solid medium is that the agar and the potassium dichromate are not added in the liquid medium, and other steps and conditions in preparation of the liquid medium are the same as those in preparation of the solid medium.

Step (2), the enrichment and cultivation of the actinomycetes is performed. 20 mL of water is taken from a water sample collected from a water source reservoir. The 20 mL water is filtered by a polyester fiber membrane with a pore size of 0.22 μm. After a filtering process is completed, the polyester filter membrane is removed and then attached to an actinomycete solid plate with sterile tweezers. The actinomycete solid pate with the polyester fiber membrane is wrapped with a sealing film. A constant temperature incubator is maintained with sufficient oxygen. The actinomycete solid plate with the polyester fiber membrane is placed upside down in the constant temperature incubator at 30° C. for 8 days until bacterial colonies develop to uniform sizes and are suitable for picking.

Step (3), white bacteria in the bacterial colonies cultured in step (2) are picked as the actinomycetes and then transferred onto the solid medium plate prepared in step (1). The actinomycetes on the solid medium plate are streaking isolated. The constant temperature incubator is maintained with sufficient oxygen. The solid medium plate with the actinomycetes is placed upside down in the constant temperature incubator at 30° C. for 8 days to culture the actinomycetes. Under sterile conditions, the actinomycetes streaking isolated are subjected to odor identification. The above operations are repeated until the actinomycetes on the solid medium plate are single in color, consistent in appearance and size, and are not accompanied by other actinomycetes, to thereby obtain a target actinomycete.

Step (4), a spore-producing medium for the actinomycetes is prepared. A formula for each liter of the spore-producing medium includes 20 g of oat flour, 0.001 g of the ferrous sulfate, 0.001 g of manganese chloride, 0.001 g of zinc sulfate, 1.29 g of calcium chloride, 0.95 g of magnesium chloride, and the ultrapure water as a solvent. The spore-producing medium is sterilized at 121° C. for 30 min in the autoclave, cooled to 55° C., and then prepared into a spore-producing medium solid plate.

Calcium and magnesium ions are key trace elements in growth of the actinomycetes. The calcium ions are essential for maintaining stability of walls of actinomycete spores, which enhances environmental adaptability of the actinomycete spores. The magnesium ions are associated with protein synthesis in the actinomycetes. A lack of the magnesium ions may lead to a reduction in a yield of the actinomycete spores. Therefore, in the disclosure, addition of these two elements aims to achieve high-quality actinomycete spore generation.

Step (5), the target actinomycete prepared in step (3) is picked and transferred onto the spore-producing medium solid plate prepared in step (4) for streaking isolation. The spore-producing medium solid plate with the target actinomycete is wrapped with the sealing film. The spore-producing medium solid plate with the target actinomycete is placed upside down in the constant temperature incubator at 30° C. for 12 days to culture the target actinomycete, to thereby obtain the actinomycete spores.

Step (6), the actinomycete spores cultured in step (5) are scraped by a sterile cotton swab. The actinomycete spores on the sterile cotton swab are suspended in a sterile phosphate-buffered solution and then vigorously oscillated with an oscillator to obtain an actinomycete spore suspension. The actinomycete spore suspension is filtered through three layers of sterile lens cleaning paper. The actinomycete spore suspension is oscillated and filtered three times to obtain a final actinomycete spore suspension. A concentration of the final actinomycete spore suspension is determined through a dilution coating plate method. The concentration of the final actinomycete spore suspension is 108 colony forming units per milliliter (CFU/mL). Finally, the final actinomycete spore suspension is mixed with sterile glycerol at a volume ratio of 1:1 in a 50 mL centrifuge tube and then stored in a refrigerator at −20° C.

Identifications are performed on the odor-producing actinomycete, including odor-producing capacity identification, molecular biology identification, spore germination determination, and morphological determination.

### 1. The Odor-Producing Capacity Identification of the Odor-Producing Actinomycete

The liquid medium for the enrichment and cultivation of the actinomycetes in step (1) of embodiment 1 is prepared. The liquid medium is sterilized together with a 250 ml conical flask. After sterilization, 100 mL of the liquid medium is added to the 250 ml conical flask. 1 mL of the final actinomycete spore suspension is added to the 250 ml conical flask. The 250 mL conical flask with the solid medium and the final actinomycete spore suspension is cultured in an oscillating incubator at a rotating speed of 130 revolutions per minute (rpm) and a temperature of 30° C. for culturing the actinomycete spores. The actinomycete spores are cultured in dark for 10 days to obtain an actinomycete bacterial liquid. 10 mL of the actinomycete bacterial liquid is extracted for odor identification.

When performing the odor identification, the actinomycete bacterial liquid needs to be pretreated, specifically including the following steps. 8 mL of the actinomycete bacterial liquid is taken as a first sample and then transferred into a first 15 mL extraction flask, and then 2.5 grams (g) of the sodium chloride and magnetic stirring bars are added to the first 15 mL extraction flask. The first 15 mL extraction flask is then quickly sealed. A fiber head of a first solid phase microextraction (SPME) head is conditioned in an injection port of a GC-MS at 250° C. until no interfering peaks are observed. The first 15 mL extraction flask is placed onto an SPME sampling device. An extraction temperature is set to 70° C., and a stirring speed is set to 500 rpm. The first 15 mL extraction flask is pre-heated on the SPME sampling device for 15 min. The first SPME head is inserted through a cap of the first 15 mL extraction flask into a headspace above the first sample. The fiber head of the first SPME head is extended into the headspace until the fiber head of the first SPME head is positioned approximately 1.0 centimeter (cm) above a surface of the first sample. The first sample is performed with headspace extraction for 20 min to obtain the fiber head with first extract of the first SPME head. After the headspace extraction is completed, the fiber head with the first extract of the first SPME head is retracted, and the first SPME head is removed from the first 15 mL extraction flask. The first SPME head is then inserted into the injection port of the GC-MS, the fiber head with the first extract of the first SPME head is extended to thermally desorb the first extract at 250° C. for 3 min, and then the first extract is injected into the GC-MS for analysis to obtain first identification results.

Parameters for the GC include a chromatographic column of DB-WAX, a length of the chromatographic column of 30 m, an inner diameter of the chromatographic column of 250 μm, a film thickness of 0.25 μm, a starting temperature of 40° C. maintained for 2 min and then raised at a rate of 10° C./min to 240° C. maintained for 3 min, a gasification chamber temperature of 250° C., a transmission line temperature of 240° C., a carrier gas of He, a velocity of the carrier gas of 1.0 mL/min, and an injection mode of splitless injection. Parameters for the MS include an ion source of EI, an electron energy of 70 eV, an ion source temperature of 230° C., a quadrupole temperature of 150° C., a scanning mode of Scan, and a scanning mass range of 35 u to 550 u.

The first identification results are performed with qualitative analysis by using a mass spectral (MS) database of National Institute of Standards and Technology 11 (NIST11) and retention time. Column loss peak is deducted from screening results of the NIST11. According to comparison between the first identification results and the NIST11, the target actinomycete produces geosmin and 2-MIB, indicating that the target actinomycete is the odor-producing actinomycete.

### 2. The Molecular Biology Identification of the Odor-Producing Actinomycete

The target actinomycete separated and purified in step (3) of embodiment 1 is performed with deoxyribonucleic acid (DNA) extraction and polymerase chain reaction (PCR) amplification sequencing, specifically including the following steps. DNA of the target actinomycete is extracted by using an Ezup column bacterial genomic DNA extraction kit according to an instruction therefor. The target actinomycete is subjected to PCR amplification by using a 16 Svedberg ribosomal DNA (16S rDNA) amplification sequence to obtain a PCR amplification product of approximately 1500 base pairs (bp). Sequences of PCR amplification primers are as follows:

27F:
recorded as SEQ ID NO: 2
AGTTTGATCMTGGCTCAG,;
and
1492R:
recorded as SEQ ID NO: 3
GGTTACCTTGTTACGACTT,.

A specific PCR amplification system includes: 0.5 microliters (μL) of Template, 2.5 μL of 10×Buffer, 1 μL of deoxy-ribonucleoside triphosphate (dNTP) with a concentration of 2.5 millimoles per liter (mM), 0.2 μL of polymerase enzyme, 0.5 μL of forward primer 27F with a concentration of 10 micromoles per liter (μM), 0.5 μL of reverse primer 1492R with a concentration of 10 μM, and double distilled water made up to a final volume of 25 μL. The Template is genomic DNA with a concentration in a range of 20 nanograms per microliter (ng/μL) to 50 ng/μL, and during amplification, the concentration of the genomic DNA is 30 ng/μL. The 10×Buffer contains divalent magnesium ions.

PCR cycling conditions include initial denaturation at 94° C. for 4 min; followed by 30 cycles of denaturation at 94° C. for 45 seconds(s), annealing at 55° C. for 45 s, and extension at 72° C. for 1 min; a final extension at 72° C. for 10 min; and reaction termination at 4° C.

The PCR amplification product is subjected to gel electrophoresis with 1% agarose, and the gel electrophoresis is performed at a voltage of 150 volts (V) and a current of 100 milliamperes (mA) for 20 min. Electrophoretic bands cut desired bands of actinomycete DNA. The PCR amplification product is directly sequenced with the PCR amplification primers to obtain a sequencing result. A sequence length of a strain of the target actinomycete is 1466 bp.

A sequence of the actinomycete is:

recorded as SEQ ID NO: 1
CAGGCTCAGGACGAACGCTGGCGGCGTGCTTAACACATGCAAGTCGA
ACGATGAAGCCCTTCGGGGTGGATTAGTGGCGAACGGGTGAGTAACA
CGTGGGCAATCTGCCCTGCACTCTGGGACAAGCCCTGGAAACGGGGT
CTAATACCGGATATCACTCTTGCAGGCATCTGTGAGGGTCGAAAGCT
CCGGCGGTGCAGGATGAGCCCGCGGCCTATCAGCTTGTTGGTGAGGT
AATGGCTCACCAAGGCGACGACGGGTAGCCGGCCTGAGAGGGCGACC
GGCCACACTGGGACTGAGACACGGCCCAGACTCCTACGGGAGGCAGC
AGTGGGGAATATTGCACAATGGGCGAAAGCCTGATGCAGCGACGCCG
CGTGAGGGATGACGGCCTTCGGGTTGTAAACCTCTTTCAGCAGGGAA
GAAGCGAAAGTGACGGTACCTGCAGAAGAAGCGCCGGCTAACTACGT
GCCAGCAGCCGCGGTAATACGTAGGGCGCAAGCGTTGTCCGGAATTA
TTGGGCGTAAAGAGCTCGTAGGCGGCTTGTCACGTCGGGTGTGAAAG
CCCGGGGCTTAACCCCGGGTCTGCATTCGATACGGGCTAGCTAGAGT
GTGGTAGGGGAGATCGGAATTCCTGGTGTAGCGGTGAAATGCGCAGA
TATCAGGAGGAACACCGGTGGCGAAGGCGGATCTCTGGGCCATTACT
GACGCTGAGGAGCGAAAGCGTGGGGAGCGAACAGGATTAGATACCCT
GGTAGTCCACGCCGTAAACGGTGGGAACTAGGTGTTGGCGACATTCC
ACGTCGTCGGTGCCGCAGCTAACGCATTAAGTTCCCCGCCTGGGGAG
TACGGCCGCAAGGCTAAAACTCAAAGGAATTGACGGGGGCCCGCACA
AGCAGCGGAGCATGTGGCTTAATTCGACGCAACGCGAAGAACCTTAC
CAAGGCTTGACATACACCGGAAACGGCCAGAGATGGTCGCCCCCTTG
TGGTCGGTGTACAGGTGGTGCATGGCTGTCGTCAGCTCGTGTCGTGA
GATGTTGGGTTAAGTCCCGCAACGAGCGCAACCCTTGTTCTGTGTTG
CCAGCATGTCCTTCGGGATGATGGGGACTCACAGGAGACCGCCGGGG
TCAACTCGGAGGAAGGTGGGGACGACGTCAAGTCATCATGCCCCTTA
TGTCTTGGGCTGCACACGTGCTACAATGGCCGGTACAAAGAGCAGCG
ATACCGTGAGGTGGAGCGAATCTCAAAAAGCCGGTCTCAGTTCGGAT
TGGGGTCTGCAACTCGACCCCATGAAGTCGGAGTTGCTAGTAATCGC
AGATCAGCATTGCTGCGGTGAATACGTTCCCGGGCCTTGTACACACC
GCCCGTCACGTCACGAAAGTCGGTAACACCCGAAGCCGGTGGCCCAA
CCCCTTGTGGGAGGGAGCTGTCGAAGGTGGGACTGGCGATTGGGACG
AAGTCGTAA,.

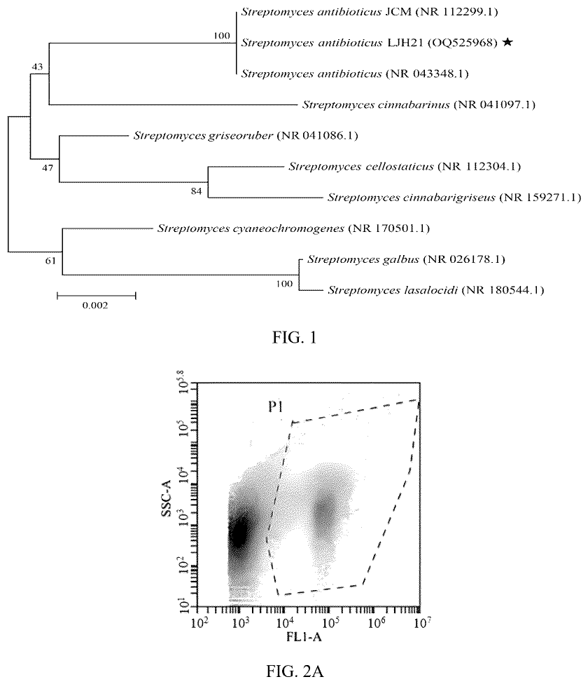

The sequencing result is submitted to National Center for Biotechnology Information (NCBI) to obtain an accession number of OQ525968 and aligned with actinomycete sequences in a GenBank database to obtain an alignment result. The alignment result is illustrated in FIG. 1, indicating that the target actinomycete has 100% similarity with Streptomyces antibioticus JCM, NR112299.1, so the target actinomycete is determined to be Streptomyces antibioticus.

### 3. The Spore Germination Determination and the Odor-Producing Capacity Identification of the Odor-Producing Actinomycete

On a basis of step (1) through step (6) in embodiment 1, 1 mL of the final actinomycete spore suspension is transferred into a 250 mL sterile conical flask loaded with 100 mL of the liquid medium prepared in step (1). The 250 mL sterile conical flask with the final actinomycete spore suspension and the liquid medium is placed in the oscillating incubator at a rotating speed of 130 rpm and a temperature of 30° C. for culturing the actinomycete spores. As illustrated in FIG. 4, during germination of the actinomycete spores from 0 h to 5 h, evenly mixed actinomycete bacterial liquid is sucked from the 250 mL sterile conical flask to a 10 mL sterile centrifuge tube every hour to obtain mixed actinomycete bacterial liquids at different times.

195 μL of the mixed actinomycetes bacterial liquids at different times are respectively taken to mix with 5 μL of stain of a flow cytometry in 2 mL sterile centrifuge tubes and then thoroughly oscillated to ensure homogeneity. The 2 mL sterile centrifuge tubes are placed in a metal bath at 35° C. and heated in the dark for 15 min to allow sufficient binding between the stain and the actinomycete to obtain heated actinomycete bacterial liquids.

Each 500 μL of the stain of the flow cytometry is prepared by dissolving 5 μL of SYBR® Green I nucleic acid stain stock solution in 495 μL of dimethyl sulfoxide. The dimethyl sulfoxide is pre-filtered by a poly tetra fluoroethylene (PTFE) membrane with a pore size of 0.2 μm. Outer walls and covers of the 2 mL sterile centrifugal tubes with the stain are wrapped with adhesive tape to avoid light, and the 2 mL sterile centrifugal tubes with the stain are stored in the refrigerator at −20° C.

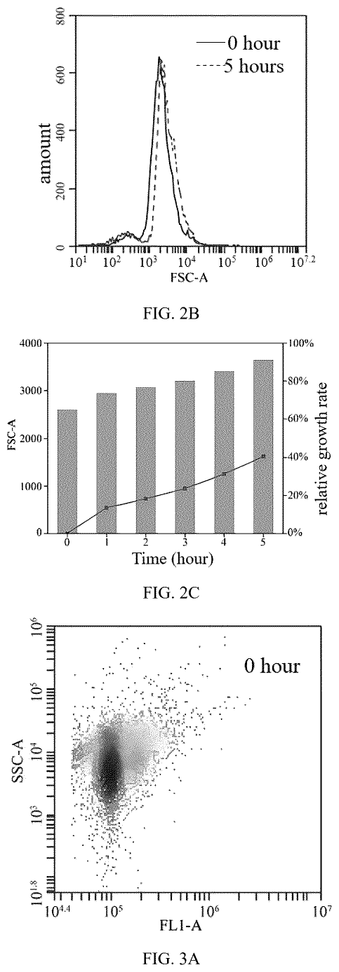

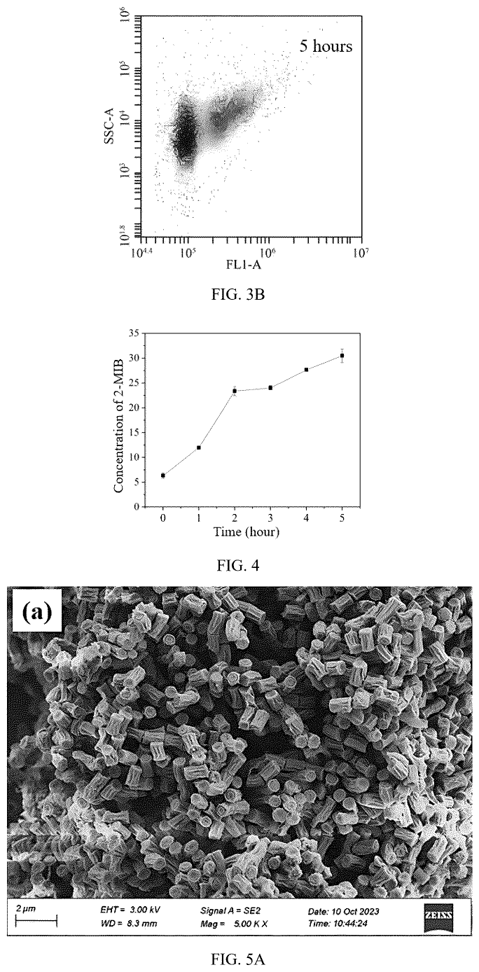

The heated actinomycete bacterial liquids are sucked into the flow cytometry, and specific gates are selected according to sampling results to distinguish broken particles from target strains, as illustrated in FIGS. 2A-2C. At the same time, a number of injected particles in each of the specific gates is set to 10000, an injection speed is medium, and a fluorescence intensity FL1-H is less than 600, in which “H” represents an intensity of electric pulse signal, is an abbreviation of “height” and represents a peak value of the electric pulse signal. Finally, parameters FSC and SSC of the flow cytometry are analyzed to determine germination state of the actinomycete spores. FSC reflects a cell particle size, SSC reflects a change of cell granularity. Overall germination speed of the actinomycete spores is reflected by a relative growth rate, namely a ratio of FSC in late germination to FSC in early germination. Change results of FSC at 0 h and 5 h are illustrated in FIGS. 2A-2C, and a change result of the SSC is illustrated in FIGS. 3A-3B.

Before measuring concentrations of odor substances, the final actinomycete spore suspension needs to be pretreated, specifically including the following steps. 8 mL of the final actinomycete spore suspension is taken as a second sample and then transferred into a second 15 mL extraction flask, 2.5 g of dried sodium chloride is added to the second 15 mL extraction flask, and then the second 15 mL extraction flask is quickly sealed. A fiber head of a second SPME head is conditioned in the injection port of the GC-MS at 250° C. until no interfering peaks are observed. The second 15 mL extraction flask is placed onto the SPME sampling device. The extraction temperature is set to 70° C., and the stirring speed is set to 500 rpm. The second 15 mL extraction flask is preheated on the SPME sampling device for 15 min. The second SPME head is inserted through a cap of the second 15 mL extraction flask into a headspace above the second sample. The fiber head of the second SPME head is extended into the headspace until the fiber head of the second SPME head is positioned approximately 1.0 cm above a surface of the second sample. The second sample is performed with headspace extraction for 20 min to obtain the fiber head with second extract. After the headspace extraction is completed, the fiber head with the second extract of the second SPME head is retracted, and the second SPME head is removed from the second 15 mL extraction flask. The second SPME head is then inserted into the injection port of the GC-MS, the fiber head with the second extract of the second SPME head is extended to thermally desorb the second extract at 250° C. for 3 min, and then the second extract is injected into the GC-MS for analysis to obtain second identification results. A change of the concentration of the 2-MIB during the germination of the actinomycete spores from 0 h to 5 h is illustrated in FIG. 4.

As illustrated in FIGS. 2A-2C, the broken particles are distinguished from the target strains by the specific gates, and fluorescence intensity FL1-A of the actinomycete spores in the specific gates changes at different times, in which “A” is an abbreviation of “area”, representing a way to read data. After 5 h of germination, an overall FSC curve of the actinomycete spores shifted to right, indicating an increase in particle size of resting spores in the liquid medium. This suggests that the actinomycete spores are transitioning from a resting state to a revived state. The FSC increases from 2594 at 0 h to 3644 at 5 h, and the relative growth rate reaches 40.5%.

As illustrated in FIGS. 3A-3B, distribution of the SSC of the actinomycete spores shows differentiation, and cell metabolism in partially revived spores becomes active, and organelles in the partially revived spores become complicated, leading to an increase of the FSC. On a contrary, resting spores remain unchanged. In addition, fluorescence intensity FL1-A of germinated spores also increases with time.

As illustrated in FIG. 4, the odor-producing actinomycete continuously produces 2-MIB during spore germination. The concentration of the 2-MIB increased from 6.3144 ng/L at 0 h to 30.4838 ng/L at 5 h, indicating that the odor-producing actinomycete has a strong ability to produce 2-MIB during the spore germination.

Microscope counting method in the art for determining the germination of the actinomycete spores has a series of disadvantages, such as uneven spore distribution, difficult discrimination, large error in results, old equipment, and so on. The aforementioned method can effectively determine the germination state of the actinomycetes spores in water through specific binding of spores and stain, high-throughput screening by the flow cytometry, rapid determination and analysis, and the relative growth rate combined with the changes of SSC and FL1-A.

### 4. The Morphological Determination of the Actinomycetes Spores

Mixed actinomycetes bacterial liquids at 0 h and 5 h are centrifuged at a rotating speed of 8000 rpm for 10 min to obtain first actinomycete spore precipitates at 0 h and 5 h and first supernatants at 0 h and 5 h. The first supernatants at 0 h and 5 h are removed. The first actinomycete spore precipitates at 0 h and 5 h are respectively added to 1.5 mL centrifuge tubes. Each of the 1.5 mL centrifuge tubes is then added with 1 mL of glutaraldehyde with a volume fraction of 2.5%. The 1.5 mL centrifuge tubes with the first actinomycete spore precipitates and the glutaraldehyde are left to stand in a refrigerator at 4° C. for 8 h. To remove excess glutaraldehyde, the first actinomycete spore precipitates in the 1.5 mL centrifuge tubes are soaked in sterile phosphate buffered saline (PBS) for 15 min for 3 times. Each time after the first actinomycete spore precipitates are soaked for 15 min, the 1.5 mL centrifuge tubes with the first actinomycete spore precipitates, the glutaraldehyde, and the sterile FBS are treated with centrifugation to obtain second actinomycete spore precipitates at 0 h and 5 h and second supernatants at 0 h and 5 h. The second supernatants are removed. After the excess glutaraldehyde is removed, excess water in the second actinomycete spore precipitates at 0 h and 5 h is removed by soaking the second actinomycete spore precipitates in a series of graded anhydrous ethanol solutions with volume concentrations of 30%, 50%, 70%, 90%, 95%, and 100%. Each time soaking the second actinomycete spore precipitates in an anhydrous ethanol solution is completed, the second actinomycete spore precipitates with the anhydrous ethanol solution is performed with centrifugation to obtain third actinomycete spore precipitates at 0 h and 5 h. The third actinomycete spore precipitates at 0 h and 5 h in the 1.5 mL centrifuge tubes are respectively added with 0.5 mL of isoamyl acetate, and then the 1.5 mL centrifuge tubes with the third actinomycete spore precipitates and the isoamyl acetate are left in a fume hood for naturally drying the third actinomycete spore precipitates to obtain final actinomycete spores at 0 h and 5 h. The final actinomycete spores at 0 h and 5 h on tube walls of the 1.5 mL centrifuge tubes are scraped, fixed with conductive adhesive, and then sprayed with gold for scanning electron microscope imaging.

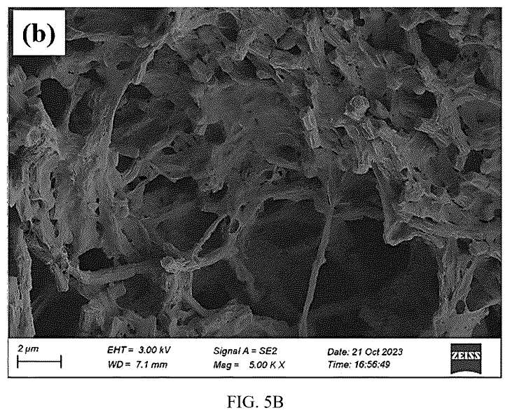

As illustrated in FIGS. 5A-5B, morphology differences of the actinomycete spores before and after germination are significant. At 0 h, the actinomycete spores are clearly observed to be arranged in order, presenting as cylindrical shapes with shriveled surfaces. However, at 5 h after germination, some of the actinomycete spores have already completed germination, formed hyphae, and adhered to the resting spores.

It should be noted that, when numerical ranges are mentioned in the claims of the disclosure, it should be understood that both endpoints of each of the numerical ranges, as well as any value within the numerical ranges between the two endpoints, can be selected. To avoid redundancy, the disclosure merely describes preferred embodiments.

Although the preferred embodiments of the disclosure have been described, those skilled in the art may make additional alterations and modifications to these embodiments once they know basic creative concepts. Therefore, the claims are intended to be construed as including the preferred embodiments and all alterations and modifications falling within the scope of protection of the disclosure.

Apparently, those skilled in the art can make various modifications and variations to the disclosure without departing from a spirit and the scope of the disclosure. Thus, it is intended that the disclosure includes these modifications and variations provided within a scope of the claims and their equivalents.

What is claimed is:

1. An odor-producing actinomycete, wherein the odor-producing actinomycete is Streptomyces antibioticus LJH21 deposit in China Center for Type Culture Collection (CCTCC) with a deposit number of CCTCC No. M 20241163.


SEQUENCE LISTING (3 sequences)

SEQ ID NO: 1 - DNA, 1466 bp, unidentified
  source 1..1466: mol_type = other DNA
caggctcaggacgaacgctggcggcgtgcttaacacatgcaagtcgaacgatgaagcccttcggggtggattagtggcgaacgggtgagtaacacgtgggcaatctgccctgcactctgggacaagccctggaaacggggtctaataccggatatcactcttgcaggcatctgtgagggtcgaaagctccggcggtgcaggatgagcccgcggcctatcagcttgttggtgaggtaatggctcaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcgggtgtgaaagcccggggcttaaccccgggtctgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaacggccagagatggtcgcccccttgtggtcggtgtacaggtggtgcatggctgtcgtcagctcgtgtcgtgagatgttgggttaagtcccgcaacgagcgcaacccttgttctgtgttgccagcatgtccttcgggatgatggggactcacaggagaccgccggggtcaactcggaggaaggtggggacgacgtcaagtcatcatgccccttatgtcttgggctgcacacgtgctacaatggccggtacaaagagcagcgataccgtgaggtggagcgaatctcaaaaagccggtctcagttcggattggggtctgcaactcgaccccatgaagtcggagttgctagtaatcgcagatcagcattgctgcggtgaatacgttcccgggccttgtacacaccgcccgtcacgtcacgaaagtcggtaacacccgaagccggtggcccaaccccttgtgggagggagctgtcgaaggtgggactggcgattgggacgaagtcgtaa

SEQ ID NO: 2 - DNA, 18 bp, unidentified
  source 1..18: mol_type = other DNA
agtttgatcmtggctcag

SEQ ID NO: 3 - DNA, 19 bp, unidentified
  source 1..19: mol_type = other DNA
ggttaccttgttacgactt